(19) United States
(12) Patent Application Publication
(10) Pub. No.: US 2013/0157334 A1
(43) Pub. Date: Jun. 20, 2013

(54) PROCESS FOR CONVERTING A LIGNOCELLULOSIC BIOMASS

(71) Applicant: Shell Oil Company, Houston, TX (US)

(72) Inventors: Evert VAN DER HEIDE, Amsterdam (NL); Albert Joseph Hendrik Janssen, Rijswijk (NL)

(73) Assignee: Shell Oil Company, Houston, TX (US)

(21) Appl. No.: 13/720,408

(22) Filed: Dec. 19, 2012

(30) Foreign Application Priority Data
Dec. 19, 2011 (EP) 11194373.4

(51) Int. Cl. C12P 7/12

(52) U.S. Cl. CPC C12P 7/12
USPC 435/164

(57) ABSTRACT

A process for converting a lignocellulosic biomass comprising
a) converting a lignocellulosic biomass into a fuel and producing an aqueous waste stream comprising at least one dissolved organic material and at least one dissolved sulfur-containing compound, wherein the aqueous waste stream has a sulfur content of more than 400 parts per million by weight, relative to the weight of the aqueous waste stream; and
b) treating the aqueous waste stream, said treating comprises anaerobic digestion of a mixture comprising a first aqueous feed comprising the aqueous waste stream and a second aqueous feed, wherein the mixture has a sulfur content of at most 400 parts per million by weight, relative to the weight of the mixture.

### CROSS-REFERENCE TO RELATED APPLICATIONS

This application claims the benefit of priority of European Patent Application No. 11194373.4, filed on Dec. 19, 2011, the disclosure of which is incorporated by reference herein in its entirety.

### FIELD OF THE INVENTION

Embodiments of the present invention relate to a process wherein a lignocellulosic biomass is converted into fuel. Embodiments of the present invention particularly relate to a process wherein a lignocellulosic biomass is converted into fuel and an aqueous waste stream is produced.

### BACKGROUND OF THE INVENTION

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

With the diminishing supply of crude petroleum oil, use of renewable energy sources is becoming increasingly important for the production of fuels. Fuels derived from non-edible renewable energy sources, such as lignocellulosic biomass, are preferred as these do not compete with food production. A process in which lignocellulosic biomass is converted into fuel, such as ethanol, may yield aqueous waste streams which are rich in dissolved organic materials and dissolved sulfur-containing compounds. In such processes lignocellulosic material may be broken up using sulfuric acid, which causes the presence of dissolved sulfur-containing compounds in the waste stream.

A suitable method for treating waste streams which comprise dissolved organic materials is by feeding the waste stream to a process for anaerobic digestion, in which the organic materials dissolved in the feed are converted by anaerobic microorganisms into a potentially valuable biogas mixture comprising methane and carbon dioxide.

Anaerobic digestion is to some extent tolerant to the presence in the feed of dissolved sulfur-containing compounds. However, in instances that the content of dissolved sulfur-containing compounds is high, anaerobic digestion may become problematic because of inhibition of the anaerobic digestion, which leads to an incomplete conversion of the dissolved organic materials and a low yield of biogas.

It has been proposed to prevent sulfide toxicity by diluting the waste stream (cf. Y. Chen, et al., “Inhibition of anaerobic digestion process: A review”, Bioresource Technology 99 (2008) 4044-4064). It has also been proposed to remove dissolved sulfur-containing compounds from the waste stream (cf., for example, S. Tait, et al., “Removal of sulfate from high-strength wastewater by crystallisation”, Water Research 43 (2009) 762-772). However, the removal of dissolved sulfur-containing compounds from the waste stream requires the use of extraneous chemicals and equipment, and is therefore considered less practicable. It has also been proposed to evaporate water from such a waste stream and to combust the residue cake; however, it is needless to say that this may be highly undesirable from an energy efficiency point of view.

It may therefore be considered an advancement in the art to provide improvements to a process for converting lignocellulosic biomass into a fuel, where an aqueous waste stream is produced, which aqueous waste stream comprises dissolved organic materials and dissolved sulfur-containing compounds.

### SUMMARY OF THE INVENTION

According to one aspect, there is provided a process comprising the steps of a) converting a lignocellulosic biomass into a fuel and producing an aqueous waste stream comprising at least one dissolved organic material and at least one dissolved sulfur-containing compound, wherein the aqueous waste stream has a sulfur content of more than 400 parts per million by weight, relative to the weight of the aqueous waste stream. The process further comprises the step of b) treating the aqueous waste stream, said treating comprises anaerobic digestion of a mixture comprising a first aqueous feed comprising the aqueous waste stream and a second aqueous feed, wherein the mixture has a sulfur content of at most 400 parts per million by weight, relative to the weight of the mixture.

In one embodiment, the second aqueous feed comprises at least one dissolved sulfur-containing compound and has a sulfur content of less than 400 parts per million by weight, relative to the weight of the second aqueous feed. In another embodiment, the second aqueous feed does not comprise a dissolved sulfur-containing compound and does not have a sulfur content. In one embodiment, the sulfur content is at most 300 parts per million by weight, relative to the weight of the second aqueous feed. In another embodiment, the sulfur content is more preferably at most 200 parts per million by weight, relative to the weight of the second aqueous feed.

In one embodiment, the dissolved organic materials comprise one or more of alcohols, monosaccharides, disaccharides, oligosaccharides, polysaccharides, aldehydes, vegetable oils, and volatile vegetable acids. In another embodiment, the first aqueous feed has a Chemical oxygen demand of at most 10⁵ mg oxygen per liter of the first aqueous feed. In another embodiment, the Chemical oxygen demand is in the range of from 5×10³ mg oxygen per liter of the first aqueous feed to 8×10⁴ mg oxygen per liter of the first aqueous feed. In another embodiment, the chemical oxygen demand is in the range of from 1×10⁴ mg oxygen per liter of the first aqueous feed to 7×10⁴ mg oxygen per liter of the first aqueous feed.

In one embodiment, step a) comprises breaking up of the lignocellulosic biomass using sulfuric acid and the at least one dissolved sulfur-containing compound present in the first aqueous feed comprises sulfates and/or sulfuric acid. In another embodiment, the sulfur content of the first aqueous feed is in the range of from 450 parts per million by weight to 4000 parts per million by weight, relative to the weight of the first aqueous feed. In another embodiment, the sulfur content of the first aqueous feed is in the range of from 500 parts per million by weight to 3000 parts per million by weight, relative to the weight of the first aqueous feed.

In one embodiment, the step of converting the lignocellulosic biomass into a fuel comprises pretreating the lignocellulosic biomass with an aqueous solution of a sulfur-containing acid and optionally with steam to produce a pretreated biomass mixture; and flashing the pretreated biomass mixture to remove water to produce a flashed pretreated biomass mixture and an aqueous flash waste stream. The aqueous flash waste stream has a sulfur content, if any, of less than 400 parts per million by weight, relative to the weight of the aqueous flash waste feed and is used as at least part of a second aqueous feed in step b). The aqueous wash waste stream comprises dissolved organic materials and dissolved sulfur-containing compounds, and has a sulfur content of more than 400 parts per million by weight, relative to the weight of the aqueous wash waste stream and is used as at least part of a first aqueous feed in step b).

In one embodiment, the process further comprises the step of washing and/or neutralizing the pretreated biomass mixture with water and/or an aqueous basic solution to produce a washed pretreated biomass mixture and an aqueous wash waste stream.

In one embodiment, the step of converting the lignocellulosic biomass into a fuel comprises pretreating the lignocellulosic biomass with an aqueous solution of a sulfur-containing acid and optionally with steam to produce a pretreated biomass mixture; and washing and/or neutralizing the pretreated biomass mixture with water and/or an aqueous basic solution to produce a washed pretreated biomass mixture and an aqueous wash waste stream.

In one embodiment, the second aqueous feed comprises sulfide salts and/or hydrogen sulfide. In another embodiment, the Chemical oxygen demand of the second aqueous feed is at most 1×10⁴ mg oxygen per liter of the second aqueous feed. In yet another embodiment, the Chemical oxygen demand is at most 8×10³ mg oxygen per liter of the second aqueous feed.

In one embodiment, the process further comprises feeding the first aqueous feed and the second aqueous feed in a relative proportion to provide a sulfur content of the resultant mixture of at most 390 parts per million by weight. In another embodiment, the sulfur content is at most 380 parts per million by weight.

In one embodiment, the process further comprises feeding the first aqueous feed and the second aqueous feed in a relative proportion to provide a weight of the second aqueous feed relative to a weight of the first aqueous feed in a range of from 20 to 0.1. In one embodiment, the range is from 15 to 0.25.

In one embodiment, the mixture comprises one or more salts of sodium, potassium, magnesium, ammonium, and any combination thereof. In one embodiment, the one or more salts has a sodium content in a range of from 50 parts per million by weight to 8000 parts per million by weight, relative to the weight of the mixture. In another embodiment, the range is from 100 parts per million by weight to 5500 parts per million by weight, relative to the weight of the mixture.

In one embodiment, the one or more salts has a potassium content a range of from 100 parts per million by weight to 12000 parts per million by weight, relative to the weight of the mixture. In another embodiment, the range is from 200 parts per million by weight to 5000 parts per million by weight, relative to the weight of the mixture.

In one embodiment, the one or more salts has a magnesium content in the range of from 40 parts per million by weight to 3000 parts per million by weight, relative to the weight of the mixture. In another embodiment, the range is from 75 parts per million by weight to 1500 parts per million by weight, relative to the weight of the mixture.

In one embodiment, the one or more salts has a content of ammonium salts in a range of from 25 parts per million by weight to 4000 parts per million by weight, relative to the weight of the mixture, wherein the content of ammonium salts relates to the quantity of ammonium salts calculated as the weight of the NH₄ moiety. In another embodiment, the range is from 50 parts per million by weight to 3000 parts per million by weight, relative to the weight of the mixture, wherein the content of ammonium salts relates to the quantity of ammonium salts calculated as the weight of the NH₄ moiety.

In one embodiment, the anaerobic digestion yields an aqueous liquid product and the process further comprises a step of removing at least a portion of the at least one dissolved sulfur-containing compound from the aqueous liquid product. In one embodiment, the removing step comprises treating the aqueous liquid product in an aerobic process. In another embodiment, the removing step yields an aqueous liquid and wherein the process further comprises recycling at least a portion of the aqueous liquid as the second aqueous feed, or as a portion of the second aqueous feed.

In one embodiment, the aqueous waste stream comprising dissolved organic materials and dissolved sulfur-containing compounds is suitably digested under the influence of anaerobic microorganisms in the presence of a second aqueous feed, which is low in its content of sulfur-containing compounds. The presence of the second aqueous feed causes the sulfur content of the digestion mixture to be low. In embodiments of this invention, favorable results are obtained in that, despite the presence in the feed of sulfur containing components at a high concentration. Certain embodiments of the invention enable the anaerobic digestion to be operated at a high conversion level of the dissolved organic materials and a high yield of biogas.

In a preferred embodiment step a) further comprises converting lignocellulosic biomass with the help of water, steam and/or an aqueous solution of a sulfur-containing acid, such as for example an aqueous solution of sulfuric acid; and at least part of any water and/or aqueous liquid obtained after the treatment in step b) is recycled for use in step a) as a portion of such water, steam and/or aqueous solution of a sulfur-containing acid. This may advantageously improve the water footprint of the process.

Other features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Embodiments of the invention may be better understood by reference to the drawing in combination with the detailed description of specific embodiments presented herein.

FIG. 1 provides a schematic of one embodiment of a treating step according to aspects of this invention.

FIG. 2 provides a schematic of one embodiment of the conversion step according to aspects of the invention.

### DETAILED DESCRIPTION OF PREFERRED EMBODIMENTS OF THE INVENTION

Embodiments of the present invention comprise a step of converting a lignocellulosic biomass into fuel and producing an aqueous waste stream. This conversion step may be referred to as “step a).” Embodiments of the present invention also comprise a step of treating the aqueous waste stream comprising anaerobic digestion. This treating step may be referred to as “step b).”

In one embodiment, in step a), the lignocellulosic biomass may be converted into one or more fuel compounds containing hydrogen and carbon atoms. The lignocellulosic biomass may be converted for example to alkanes and/or alkenes, (such as for example C5-C18 alkanes and/or alkenes; by a Cx-Cy compound is herein understood a compound containing equal to or more than x and equal to or less than y carbon atoms); or for example to one or more alkanols and or fatty acids (such as for example C2-C18 alkanols and or C2-C18 fatty acids). In a preferred embodiment, the lignocellulosic biomass is converted into one or more alkanols, such as for example ethanol and/or butanol.

The term “a lignocellulosic biomass” is herein understood to refer at least to a material containing cellulose and lignin and optionally hemicellulose. The lignocellulosic biomass may herein also be referred to as lignocellulosic material. The lignocellulosic biomass may be obtained from a wide variety of sources, including for example plants, forestry residues, agricultural residues, herbaceous material, municipal solid wastes, waste and recycled paper, pulp and paper mill residues, sugar processing residues and/or combinations of one or more of the above.

The lignocellulosic biomass can comprise for example, corn stover, soybean stover, corn cobs, corn fibre, straw (including cereal straws such as wheat, barley, rye and/or oat straw), bagasse, beet pulp, miscanthus, sorghum residue, rice straw, rice hulls, oat hulls, grasses (including switch grass, cord grass, rye grass, reed canary grass or any combination thereof), bamboo, water hyacinth, wood and wood-related materials (including hardwood, hardwood chips, hardwood pulp, softwood, softwood chips, softwood pulp and/or sawdust), waste paper and/or any combination of one or more of these.

In a preferred embodiment, the lignocellulosic biomass may be converted into fuel with the help of a sulfur-containing acid. Examples of sulfur-containing acids that can be used in step a) include sulfuric acid and sulfurous acid. Preferably the lignocellulosic biomass is converted into a fuel with the help of sulfuric acid. Conveniently, the lignocellulosic biomass may be broken up using sulfuric acid. In a preferred embodiment, the lignocellulosic biomass is converted into fuel with the help of an aqueous solution of the sulfur-containing acid, more preferably an aqueous solution of sulfuric acid is used. The use of a sulfur-containing acid in step a) may lead to the presence of a high content of dissolved sulfur-containing compounds in the aqueous waste stream.

In a preferred embodiment, step a) further comprises converting lignocellulosic biomass in the presence of water. The water in step a) may for example be present in the form of steam and/or in the form of an aqueous solution, such as the aqueous solution of a sulfur-containing acid. In step a), steam may conveniently be used to regulate the temperature.

In one preferred embodiment, step a) comprises converting lignocellulosic biomass into a fuel by a method comprising:

pretreating the lignocellulosic biomass with an aqueous solution of a sulfur-containing acid and optionally with steam to produce a pretreated biomass mixture;
flashing the pretreated biomass mixture to remove water to produce a flashed pretreated biomass mixture and an aqueous flash waste stream; and/or
washing and/or neutralizing the pretreated biomass mixture with water and/or an aqueous basic solution to produce a washed pretreated biomass mixture and an aqueous wash waste stream. The pretreatment, flashing and washing step may be carried out as described in more detail hereinbelow.

In this embodiment, the aqueous flash waste stream and/or the aqueous wash waste stream may comprise dissolved organic materials and dissolved sulfur-containing compounds, and may have a sulfur content of more than 400 parts per million by weight, relative to the weight of the aqueous waste stream. If both the aqueous flash waste stream and the aqueous wash waste stream have a sulfur content of more than 400 parts per million by weight, it may be advantageous to combine the aqueous flash waste stream and the aqueous wash waste stream and use this combination as a first aqueous feed or as part of a first aqueous feed in step b). In a preferred embodiment, the aqueous flash waste stream may have a sulfur content, if any, of less than 400 parts per million by weight, relative to the weight of the aqueous flash waste stream. When the aqueous flash waste stream has a sulfur content, it is less than 400 parts per million by weight, relative to the weight of the aqueous flash waste stream. The aqueous flash stream may conveniently be used as a second aqueous feed or as part of a second aqueous feed in step b). In another preferred embodiment, the aqueous flash waste stream may have a sulfur content, if any, of less than 400 parts per million by weight, relative to the weight of the aqueous flash waste stream, and may conveniently be used as a second aqueous feed or as part of a second aqueous feed in step b). The aqueous wash waste stream may comprise dissolved organic materials and dissolved sulfur-containing compounds, and the aqueous wash waste stream may have a sulfur content of more than 400 parts per million by weight, relative to the weight of the aqueous waste stream, and may be used as a first aqueous feed or as part of a first aqueous feed in step b). In such an embodiment the aqueous wash waste stream and aqueous flash waste stream may advantageous be combined to form the mixture in step b). That is, the mixture in step b) may comprise the aqueous wash waste stream as a first aqueous feed and the aqueous flash waste stream as a second aqueous feed.

In another preferred embodiment, step a) comprises converting lignocellulosic biomass into one or more alkanols by a method comprising:

i) pretreating the lignocellulosic biomass with an aqueous solution of sulfuric acid and optionally with steam at a temperature in the range from equal to or more than 100° C. to equal to or less than 250° C. to produce a pretreated biomass mixture; and/or

ii) optionally flashing the pretreated biomass mixture to remove water to produce a flashed pretreated biomass mixture and an aqueous flash waste stream; and/or

iii) optionally washing and/or neutralizing part or whole of the pretreated biomass mixture to produce a washed and/or neutralized pretreated biomass mixture and an aqueous wash waste stream; and/or

iv) hydrolysis of part or whole of the, optionally washed and/or neutralized, pretreated biomass mixture to produce a hydrolysis product; and/or

v) fermentation of part or whole of the hydrolysis product to produce a fermentation mixture; and/or

vi) separating the fermentation mixture into one or more alkanol(s) and an aqueous fermentation waste stream. These steps may be referred to as “step i),” “step ii),” “step iii),” “step iv,” “step v,” and “step vi,” respectively.

The aqueous flash waste stream from step ii); the aqueous wash waste stream from step iii) and/or the aqueous fermentation waste stream from step vi) may comprise dissolved organic materials and dissolved sulfur-containing compounds, and may have a sulfur content of more than 400 parts per million by weight, relative to the weight of the aqueous waste stream. In one embodiment, one, or a combination of two or more, of the aqueous waste streams generated in steps ii), iii) or step vi) or a combination of both of the aqueous waste streams generated in step iii) and vi) can be forwarded to step b) as a first aqueous feed.

Alternatively, when the aqueous flash waste stream from step ii) has a sulfur content, if any, of less than 400 parts per million by weight, relative to the weight of the aqueous flash waste stream, the aqueous flash stream may conveniently be used as a second aqueous feed in step b). In this case the aqueous flash waste stream from step ii) may conveniently be combined with the aqueous wash waste stream from step iii) and/or the aqueous fermentation waste stream from step vi) to make a mixture as mentioned for step b). That is, the mixture in step b) may comprise the aqueous wash waste stream from step iii) and/or the aqueous fermentation waste stream from step vi) as a first aqueous feed; and the aqueous flash waste stream from step ii) as a second aqueous feed.

Prior to pretreating in step a), the lignocellulosic biomass can be washed and/or reduced in particle size. The particle size reduction may for example include grinding, chopping, crushing or debarking of a lignocellulosic biomass. In a preferred embodiment, the particle size of the lignocellulosic biomass is reduced to a particle size in the range from equal to or more than 5 micron to equal to or less than 5 cm, more preferably in the range from 2 mm to 10 mm.

In another preferred embodiment, the pretreating of step a) comprises contacting the lignocellulosic biomass at a temperature in the range from equal to or more than 100° C., more preferably equal to or more than 120° C., even more preferably equal to or more than 160° C., to equal to or less than 250° C., more preferably to equal to or less than 230° C., even more preferably to equal to or less than 210° C. with an aqueous solution of sulfuric acid. In a preferred embodiment, such an aqueous solution of sulfuric acid may be prepared whilst using aqueous liquid recycled from step b), as described in more detail herein below. The aqueous solution of sulfuric acid preferably has a pH in the range from equal to or more than 0.0 to equal to or less than 4.5, more preferably in the range from equal to or more than 0.5 to equal to or less than 2.0. For practical purposes the pressure preferably lies in the range from equal to or more than an atmospheric pressure of about 0.1 MegaPascal (MPa) to equal to or less than 3.0 MPa. After pretreatment, a pretreated biomass mixture can be obtained.

The pretreated biomass mixture may be forwarded to a subsequent step as a whole or only in part. For example, if so desired, an aqueous waste stream (herein also referred to as aqueous flash waste stream) may be removed from the pretreated biomass mixture, for example by one or more flashing steps and/or one or more distillation steps. As explained above an aqueous waste stream obtained by flashing or distillation of the pretreated biomass mixture may suitably be used as first aqueous feed or part thereof in step b); or as second aqueous feed or part thereof in step b). As the sulfur content of the aqueous waste stream obtained after flashing and/or distillation of the pretreated biomass mixture may be low, it is most advantageous to use this aqueous waste stream as second aqueous feed in step b).

If so desired, at least part of or the whole of the pretreated biomass mixture may be washed and/or neutralized. For example at least part of the pretreated biomass mixture may be washed and/or neutralized with water and/or with an aqueous basic solution to a pH in the range from equal to or more than 4.0 to equal to or less than 7.0. In a preferred embodiment, the washed and/or neutralized pretreated biomass mixture has a pH in the range from equal to or more than 4.0 to equal to or less than 7.0, more preferably in the range from equal to or more than 4.5 to equal to or less than 6.0. Particularly, the aqueous liquid can be recycled from step b) and can be used as the water and/or in the preparation of the aqueous basic solution as described in more detail herein below. The aqueous waste stream(s) (herein also referred to as aqueous wash waste stream) that may be obtained during washing and/or neutralizing, may contain dissolved sulfur-containing compounds resulting from the use of sulfuric acid. In addition such aqueous waste stream(s) may contain dissolved organic materials such as one or more sugars (for example xylose, galactose, mannose, glucose, arabinose) and/or sugar dimers and/or sugar polymers (such as for example xylan, arabinoxylan, glucoronoxylan, xyloglucan). These aqueous waste stream(s) may therefore advantageously be forwarded to step b) as the first aqueous feed or part thereof for treatment in step b). The dissolved organic materials mentioned above, such as the sugars and/or sugar dimers and/or sugar polymers may conveniently be converted into methane in step b).

In a preferred embodiment, the optionally washed and/or neutralized, pretreated biomass mixture is subsequently hydrolyzed to produce a hydrolysis product. The hydrolysis may be carried out in any manner known to the skilled person in the art. In another preferred embodiment, part or all of the optionally washed and/or neutralized, pretreated biomass mixture is hydrolyzed in step iv) by enzymatic hydrolysis. In a particular preferred embodiment, the hydrolysis comprises hydrolyzing part or all of the optionally neutralized, pretreated biomass mixture with the help of one or more cellulase enzymes. A cellulase enzyme (also sometimes referred to as “cellulase”) can catalyze the hydrolysis of cellulose present in the optionally neutralized, pretreated biomass mixture. The cellulase enzyme may be any cellulase enzyme known to the skilled person to be suitable for hydrolysis of cellulose. Examples of suitable cellulase enzymes include cellulase enzymes obtained from fungi of the genera Aspergillus, Humicola and Trichoderma and/or Myceliophthora and from the bacteria of the genera Bacillus and Thermobifida. Examples of the cellulase enzymes include cellobiohydrolases (CBH's), endoglucanases (EG's), beta-glucosidases and mixtures thereof. In addition to cellulase enzymes, hemicellulase enzymes, esterase enzymes and swollenins may be present. The cellulase enzyme dosage may for example be in the range from 3.0 to 100.0 Filter Paper Units (FPU or IU) per gram of cellulose. The FPU is a standard measurement and is defined and measured according to Ghose (1987, Pure and Appl. Chem. 59: pages 257-268). In a preferred embodiment, any enzymatic hydrolysis in step iv) is carried out at a temperature of equal to or more than 15° C., more preferably equal to or more than 20° C. and most preferably equal to or more than 25° C. whilst the temperature is preferably equal to or less than 80° C., more preferably equal to or less than 70° C. and most preferably equal to or less than 55° C. Most preferably the enzymatic hydrolysis is carried out at a temperature in the range from equal to or more than 25° C. to equal to or less than 55° C.

In another preferred embodiment, the enzymatic hydrolysis is carried out for a reaction time equal to or more than 1 hour, more preferably equal to or more than 5 hours, even more preferably equal to or more than 10 hours. And preferably the enzymatic hydrolysis is carried out for a reaction time equal to or less than 300 hours, more preferably equal to or less than 200 hours, most preferably equal to or less than 100 hours. Most preferably the enzymatic hydrolysis is carried out for a reaction time in the range from equal to or more than 24 hour to equal to or less than 72 hours.

In one embodiment, the hydrolysis step iv) produces a hydrolysis product. The hydrolysis product may contain one or more sugars. The sugars may comprise for example monosaccharides and disaccharides. For example the hydrolysis product may contain glucose, xylose, galactose, mannose, arabinose, fructose, rhamnose and/or mixtures thereof.

Where step iv) produces an effluent containing a liquid hydrolysis product and one or more solids, the liquid hydrolysis product may be separated from such one or more solids by means of a liquid/solid separation.

Part or all of the hydrolysis product may be fermented to produce a fermentation mixture. The fermentation in step v) may for example be carried out with the help of a microorganism. In a preferred embodiment, the microorganism is a microorganism capable of fermenting part or all of the hydrolysis product to a fermentation mixture containing ethanol and/or butanol. In one embodiment, the microorganism is chosen from the group consisting of Saccharomyces spp., Saccharomyces cerevisiae, Escherichia, Zymomonas, Candida, Pichia, Streptomyces, Bacillus, Lactobacillus, Clostridium and any combination thereof.

In a preferred embodiment, the fermentation in step v) is carried out at a temperature of equal to or more than 15° C., more preferably equal to or more than 20° C. and most preferably equal to or more than 25° C. whilst the temperature is preferably equal to or less than 50° C., more preferably equal to or less than 40° C. and most preferably equal to or less than 35° C.

In another preferred embodiment, the fermentation in step v) is carried out at a pH in the range from equal to or more than 3.0 and equal to or less than 6.0, more preferably in the range from equal to or more than 4.0 to equal to or less than 6.0. If desired oxygen and/or one or more additional nutrients for the microorganism may be added to step v). Examples of additional nutrients are yeast extract, specific amino acids, phosphate, nitrogen sources, salts, trace elements and vitamins.

The fermentation may be carried out in batch, continuous or fed-batch mode with or without agitation. The fermentation may be carried out in one or more reactors, preferably in a series of 1 to 6 fermentation reactors. Preferably the fermentation is carried out in one or more mechanically stirred reactors. The fermentation microorganisms may be recycled back to the fermentation reactor. Or they may for example be sent to distillation without recycle.

In one embodiment the hydrolyzing of step iv) and the fermentation of step v) are carried out simultaneously in the same reactor. It is, however, most preferred to carry out the hydrolyzing of step iv) and the fermentation of step v) separately to allow for optimal temperatures for each step.

The fermentation mixture suitably generated in step v) may contain one or more alkanols. Preferably the fermentation mixture contains ethanol and/or butanol. Most preferably the fermentation mixture is a fermentation mixture containing ethanol. In addition the fermentation mixture may contain water and/or solids. Examples of solids that may be present in the fermentation mixture include unconverted pretreated lignocellulosic biomass, lignin and/or any solid components added during fermentation.

Where step v) produces a fermentation mixture containing a liquid and one or more solids, the solids are preferably removed from the fermentation mixture by means of a liquid/solid separation.

In step vi), the fermentation mixture may be separated into one or more alkanol(s) and an aqueous waste stream (herein also referred to as aqueous fermentation waste stream). This aqueous waste stream may comprise dissolved organic materials and dissolved sulfur-containing compounds, and may have a sulfur content of more than 400 parts per million by weight, relative to the weight of the aqueous waste stream. As explained herein before this aqueous fermentation waste stream may therefore conveniently be used as first aqueous feed or part thereof in step b).

The one or more alkanols and the aqueous waste stream may suitably be retrieved from the fermentation mixture by distillation of the fermentation mixture to produce one or more distillation fraction(s) comprising the one or more alkanol(s), and one or more distillation fraction(s) comprising the aqueous waste stream.

In a process according to aspects of the invention, an aqueous waste stream obtained from step a) is used as a first aqueous feed in step b). The first aqueous feed in step b) may for example comprise an aqueous flash waste stream, an aqueous wash waste stream, an aqueous fermentation waste stream and/or any combination thereof. One skilled in the art will therefore understand that all preferences for the first aqueous feed as described below may also apply to the one or more aqueous waste stream(s) obtained in step a) and used in step b) as first aqueous feed.

Preferred processes for converting lignocellulosic biomass into ethanol are described in for example U.S. Pat. No. 4,612,286, US 2011/0281298-A1, WO 2011/022840 A1 and WO 2011/084761 A2. In such a process, ethanol may be recovered by distillation from an aqueous fermentation mixture. The bottom product of the distillation may be a waste stream which may suitably be applied as the first aqueous feed in step b).

Step b) employs an aqueous waste stream from step a) as a first aqueous feed. As explained above the aqueous waste stream from step a) may comprise for example an aqueous flash waste stream, an aqueous wash waste stream, an aqueous fermentation waste stream and/or any combination thereof (for example it may contain an aqueous waste stream from step ii), step iii), step vi) or any combination thereof). Most preferably the aqueous waste stream from step a) used as first aqueous feed in step b) comprises an aqueous wash waste stream, an aqueous fermentation waste stream and/or any combination thereof (for example it may contain an aqueous waste stream from step iii), step vi) or any combination thereof).

The first aqueous feed comprises organic materials and sulfur-containing compounds dissolved in water. The dissolved organic materials may comprise organic compounds such as, for example, alcohols, such as ethanol and n-propanol; monosaccharides, such as arabinose, glucuronic acid, galacturonic acid, mannose, galactose, glucose, xylose and fructose; disaccharides, such as sucrose and cellobiose; oligosaccharides, such as glucans and xylans; polysaccharides, such as celluloses, hemicelluloses, xylan, glucan and starch; aldehydes, such as furfural and hydroxymethyl furfural; vegetable oils, such as tall oil, fatty acid triglycerides and fatty acids; and volatile vegetable acids, such as C₁ to C₅ carboxylic acids (inclusive), including formic acid, acetic acid, propionic acid, butyric acid and pentanoic acid. The quantity of organic materials present in the first aqueous feed may preferably be such that the Chemical oxygen demand (“COD”, hereinafter) of the first aqueous feed may be up to 10⁵ mg oxygen per liter of the first aqueous feed, in particular in the range of from 5×10³ mg oxygen per liter of the first aqueous feed to 8×10⁴ mg oxygen per liter of the first aqueous feed, more in particular in the range of from 1×10⁴ mg oxygen per liter of the first aqueous feed to 7×10⁴ mg oxygen per liter of the first aqueous feed. As used herein, COD is as measured by the method of ISO 6060, using potassium dichromate as the oxidant.

The dissolved sulfur-containing compounds may generally comprise sulfur containing inorganic salts, such as sulfates, sulfites and sulfides, and the corresponding acids. The dissolved sulfur-containing compounds present in the first aqueous feed may preferably comprise sulfates and/or sulfuric acid.

As used herein, organic compounds are generally compounds comprising one or more covalent C—H bonds in their molecular structure, and inorganic compounds, for example inorganic salts, are generally compounds not comprising covalent C—H bonds in their molecular structure.

The sulfur content of the first aqueous feed may be in the range of from more than 400 parts per million by weight (ppmw) to 5000 ppmw, relative to the weight of the first aqueous feed. More preferably, the sulfur content of the first aqueous feed may be in the range of from 450 ppmw to 4000 ppmw, in particular in the range of from 500 ppmw to 3000 ppmw, relative to the weight of the first aqueous feed. In general, the dissolved sulfur-containing compounds present in the first aqueous feed contribute to the sulfur content of the first aqueous feed. The first aqueous feed may or may not comprise to some extent dissolved organic materials which comprise sulfur atoms in their molecular structure, in which case the sulfur present in the sulfur containing organic materials also contribute to the sulfur content of the first aqueous feed. As used herein, sulfur content relates to the quantity of sulfur calculated as elemental sulfur; sulfur content may be as determined by ASTM D1976, modified in that, if the pH of the sample to be analysed is lower than 10, aqueous sodium hydroxide is added to the sample to increase the pH of the sample to at least 10. As used herein, pH is as measured at 20° C.

The pH of the first aqueous feed may preferably be slightly basic. Preferably, the pH of the first aqueous feed may be at most 10, more preferably in the range of from 7 to 9, preferably in the range of from 7.5 to 8.5.

The first aqueous feed may or may not comprise solids, such as particles of lignin, sand or clay. Preferably, any solid particles may be present to a minor extent, such that the first aqueous feed is still pumpable. It is preferred to have solid particles removed from the first aqueous feed, for example by filtration or centrifugation.

The second aqueous feed is preferably water. The second aqueous feed is most preferably clean water, although impurities may be present. For example, the second aqueous feed may or may not comprise dissolved sulfur-containing compounds. If dissolved sulfur-containing compounds are present, the second aqueous feed may preferably have a sulfur content of less than 400 ppmw, relative to the weight of the second aqueous feed. If dissolved sulfur-containing compounds are present, they may preferably comprise sulfate salts and/or sulfite salts and/or the corresponding acids. If dissolved sulfur-containing compounds are present, they may in particular comprise sulfide salts and/or hydrogen sulfide. Preferably, the second aqueous feed has a sulfur content of at most 300 ppmw, more preferably at most 200 ppmw, relative to the weight of the second aqueous feed. In one embodiment, the second aqueous feed may have a sulfur content of at least 10 ppmw, or at least 1 ppmw, relative to the weight of the second aqueous feed. Dissolved organic materials, such as specified hereinbefore, may or may not be present in the second aqueous feed. The dissolved organic materials may be present in a quantity as specified hereinbefore in connection with the first aqueous feed.

In another preferred embodiment, in particular in cases that the second aqueous feed comprises an aqueous liquid obtained in, and recycled from, a process according to aspects of the invention, as described hereinafter, that the quantity of the dissolved organic materials present in the second aqueous feed is such that the COD of the second aqueous feed is at most 1×10⁴ mg oxygen per liter of the second aqueous feed, more preferably at most 8×10³ mg oxygen per liter of the second aqueous feed. In one embodiment, the COD of the second aqueous feed may be at least 10 mg oxygen per liter of the second aqueous feed, or at least 1 mg oxygen per liter of the second aqueous feed.

The second aqueous feed may or may not comprise solids, such as particles of lignin, sand or clay. Solid particles may be present to a minor extent, such that the second aqueous feed is still pumpable. It is preferred to have solid particles removed from the second aqueous feed, for example by filtration or centrifugation.

In an especially preferred embodiment, the second aqueous feed may be essentially pure water. That is, in an especially preferred embodiment the second aqueous feed contains less than 10 ppmw, more preferably less than 1 ppmw dissolved sulfur-containing compounds and less than 10 ppmw, more preferably less than 1 ppmw dissolved organic materials, relative to the weight of the second aqueous feed.

The second aqueous feed may comprise a waste stream of a plant for processing, for example, fruit, vegetables, agricultural waste, forest waste or municipal waste. Alternatively or additionally, the second aqueous feed may comprise water taken from a river or a lake, or ground water. In one preferred embodiment, the second aqueous feed comprises an aqueous flash waste stream (for example the aqueous waste stream obtained from step ii) as described herein above, where this aqueous flash waste stream contains less than 400 parts per million by weight, relative to the weight of the aqueous flash waste stream.

In another preferred embodiment, the second aqueous feed may partially or entirely comprise an aqueous liquid obtained in, and recycled from, a process according to aspects of the invention, as described hereinafter. In this preferred embodiment there is the additional advantage that the net quantity of liquid product produced in the process is essentially equal to the quantity of the first aqueous feed, and the net quantity of liquid product produced in the process is not essentially increased by feeding the second aqueous feed to process.

The first aqueous feed and the second aqueous feed may be fed to the process of this invention in such a relative proportion that the sulfur content of the resultant mixture is at most 400 ppmw. Preferably the sulfur content of the resultant mixture may be at most 390 ppmw, more preferably at most 380 ppmw. In one embodiment, the sulfur content of the resultant mixture may suitably be at least 20 ppmw, or at least 10 ppmw. Preferably, the weight of the second aqueous feed relative to the weight of the first aqueous feed, as fed to the process, may be in the range of from 20 to 0.1, more preferably in the range of from 15 to 0.25.

The first aqueous feed and the second aqueous feed may be fed separately to a process according to aspects of the invention, and form the mixture within the process. Alternatively, or in addition, the first aqueous feed and the second aqueous feed may be mixed and subsequently fed to a process according to aspects of the invention.

The pH of the mixture may preferably be slightly basic. Preferably, the pH of the mixture may be at most 10, more preferably in the range of from 7 to 9, preferably in the range of from 7.5 to 8.5. Dependent of the conditions which are optimal for digestion under the influence of the anaerobic microorganisms, the pH may be adjusted by adding an acid, for example hydrochloric acid, or a base, for example sodium carbonate or sodium hydroxide.

Preferably, the sodium content of the mixture may be in the range of from 50 ppmw to 8000 ppmw, more preferably in the range of from 100 ppmw to 5500 ppmw, relative to the weight of the mixture. As used herein, sodium content relates to the quantity of sodium calculated as the weight of sodium metal, sodium content may be as determined by ASTM D1976.

Preferably, the potassium content of the mixture may be in the range of from 100 ppmw to 12000 ppmw, more preferably in the range of from 200 ppmw to 5000 ppmw, relative to the weight of the mixture. As used herein, potassium content relates to the quantity of potassium calculated as the weight of potassium metal, potassium content may be as determined by ASTM D1976.

Preferably, the magnesium content of the mixture may be in the range of from 40 ppmw to 3000 ppmw, more preferably in the range of from 75 ppmw to 1500 ppmw, relative to the weight of the mixture. As used herein, magnesium content relates to the quantity of magnesium calculated as the weight of magnesium metal, magnesium content may be as determined by ASTM D1976.

Preferably, the content of ammonium salts of the mixture may be in the range of from 25 ppmw to 4000 ppmw, more preferably in the range of from 50 ppmw to 3000 ppmw, relative to the weight of the mixture. As used herein, the content of ammonium salts relates to the quantity of ammonium salts calculated as the weight of the NH₄ moiety, the content of ammonium salts may be as determined by ASTM D1426-08, in particular method B therein.

Contents of sodium, potassium, magnesium and ammonium salts as specified in the preceding paragraphs tend to provide stimulatory activity of the respective salts in the anaerobic digestion and/or tend to prevent inhibitory effects.

Process conditions of the anaerobic digestion may for example be as described in U.S. Pat. No. 4,551,250; E. ten Brummeler, et al., “Dry Anaerobic Batch Digestion of the Organic Fraction of Municipal Solid Waste”, J. Chem. Tech. Biotechnol. 50 (1991), pp. 191-209; and J. B. van Lier, et al., Thermo-Tolerant Anaerobic Degradation of Volatile Fatty Acids by Digested Organic Fraction of Municipal Solid Waste”, Journal of Fermentation and Bioengineering, 76, No. 2 (1993) pp. 140-144.

Preferably, in accordance with the present invention the process of anaerobic digestion involves the presence of anaerobic microorganisms, in particular acetic acid forming bacteria, also referred to as acetogens; methane forming archaea, also referred to as methanogens; and sulfate reducing bacteria. Suitable anaerobic microorganisms are ubiquitous in municipal sludge digestion, or they may be purchased from vendors, such as Paques B. V. (T. de Boerstraat 24, 8561EL Balk, The Netherlands). A useful source of the anaerobic microorganisms may be taken as biosludge from an existing water treatment plant, for example a plant for treating municipal waste. The anaerobic microorganisms may preferably be added to the process in the form of a granular sludge. The microorganisms which are best acclimated to the substrate and reaction conditions will prevail and sustain the desired anaerobic digestion.

In the anaerobic digestion, the temperature is preferably in the range of from 10° C. to 100° C. The anaerobic microorganisms may preferably be mesophiles or thermophiles.

In the case that the anaerobic microorganisms are mesophiles, the temperature of the anaerobic digestion is preferably kept in the range of from 15° C. to 45° C., more preferably in the range of from 20° C. to 40° C., in particular in the range of from 25° C. to 35° C. In the case that the anaerobic microorganisms are thermophiles, the temperature of the anaerobic digestion is preferably kept in the range of from 40° C. to 75° C., more preferably in the range of from 45° C. to 70° C., in particular in the range of from 50° C. to 65° C. The use of mesophilic microorganisms is preferred as these provide a more stable operation performance. The pressure maintained during the anaerobic digestion may preferably be in the range of from 80 kiloPascal (kPa) to 200 kPa, more preferably in the range of from 90 kPa to 150 kPa. As used herein, pressure is absolute pressure.

The process of anaerobic digestion may be carried out batch wise, or as a continuous process, for example in a one or more stirred tank reactor. In a continuous process, a plurality of stirred tank reactors may be arranged in series or parallel. In a continuous process, an upflow anaerobic sludge blanket (UASB) reactor or an expanded granular sludge blanket (EGSB) reactor may be employed. Alternatively, a BIOCEL reactor may be employed. Such reactors are commonly known in the art, for example, from the references provided hereinbefore. The total residence time of the aqueous phase in the process of anaerobic digestion may preferably be in the range of from 1 day to 40 days (inclusive), more preferably in the range of from 2 days to 20 days (inclusive).

The product obtained from the process of anaerobic digestion comprises a liquid, with solids suspended therein and a gas. Solids may be removed from the liquid filtration or centrifugation. However, preferably, solids are allowed to settle and removed by decantation.

Suitably the liquid product obtained from the process of anaerobic digestion is an aqueous liquid comprising dissolved sulfur-containing compounds. The dissolved sulfur-containing compounds preferably comprise sulfide salts and/or hydrogen sulfide. The liquid product obtained from the process of anaerobic digestion may or may not comprise dissolved organic materials. If present, the dissolved organic materials may preferably be present in a quantity such that the COD may be up to 5×10³ mg oxygen per liter of the first aqueous feed, in particular in the range of from 2×10² mg oxygen per liter of the first aqueous feed to 2×10³ mg oxygen per liter of the first aqueous feed, more in particular in the range of from 5×10² mg oxygen per liter of the first aqueous feed to 1.5×10³ mg oxygen per liter of the first aqueous feed.

In a further or alternative preferred embodiment at least part of the liquid product obtained from the process of anaerobic digestion may be treated further, to at least partially remove the dissolved sulfur-containing compounds (hereinafter referred to as “sulfur removing step”).

The sulfur removing step may preferably comprise an aerobic process, known from for example WO 91/16269 A1 and WO 2005/044742 A1, and sometimes referred to as the Shell-Paques process (cf. A. J. H. Janssen et al., “Application of bacteria involved in the biological sulfur cycle for paper mill effluent purification”, Science of the Total Environment, 407 (2009) 1333-1343; and “Test and Quality Assurance Plan; Paques THIOPAQ and Shell Paques Gas Purification Technology”, Report prepared by Greenhouse Gas Technology Center Southern Research Institute (PO Box 13825, Research Triangle Park, North Carolina 27709, USA), under a cooperation agreement with U.S. Environmental Protection Agency, Southern/USEPA-GHG-QAP-32, June 2004). In this process, the dissolved sulfur-containing compounds are at least partially oxidized in a bioreactor to form elemental sulfur, which oxidation may be catalyzed by microorganisms of the genus Thiobacillus or Halothiobacillus. For start-up, the bioreactor may be occulated with up to 5%, in particular 1%, of its wet volume with a bio sulfur slurry from an existing installation, whereafter the sulfur loading may be increased stepwise. Preferably, the oxidant applied is air, which may be blown into the bioreactor to enhance mixing. The pressure in the bioreactor may preferably be in the range of from 90 kPa to 110 kPa, more preferably in the range of from 95 kPa to 105 kPa. The temperature in the bioreactor may preferably be maintained at a value in the range of from 15° C. to 48° C., more preferably in the range of from 25° C. to 40° C. The pH may preferably be maintained at a value in the range of from 7 to 9.5, more preferably in the range of from 8 to 9. The bacteria may be maintained by adding a combination of nutrients, for example comprising, per liter, 4 g of ammonium chloride, 1 g of magnesium sulfate as MgSO₄.7H₂0, 2 g of potassium dihydrogen phosphate and 10 ml of a trace element solution according to Vishniac and Santer, “The Thiobacilli”, Bacteriol. Rev. 21 (1957) 195-213. This combination of nutrients may be fed to the bioreactor at a rate of at most 1.4 kg of the nutrient solution per kg of sulfide to be converted, calculated as the weight of elemental sulfur. When the liquid product obtained from the process of anaerobic digestion comprises nutrients, less of the nutrients may be fed to the bioreactor accordingly. Elemental sulfur formed may be separated from the remaining aqueous liquid by means of a gravity separator or a decanter centrifuge. The slurry so obtained may be employed as bio slurry sulfur at start-up, as described hereinbefore. The gravity separator may be positioned inside or outside the bioreactor.

If any organic material is present in the liquid product obtained from the process of anaerobic digestion, a portion thereof may be oxidized in the bioreactor in which dissolved sulfur-containing compounds are to form elemental sulfur. For the oxidation of any remaining organic material and for the removal of residual sulfur particles, if any, a treatment in an aerated sand filter may be adequate.

The aqueous liquid obtained from the sulfur removing step, or a portion thereof, may be recycled and used as the second aqueous feed, or as a portion of the second aqueous feed, as described hereinbefore. As an alternative, the aqueous liquid obtained in the further treatment described in the preceding paragraph may be recycled and used as the second aqueous feed, or as a portion of the second aqueous feed.

In one preferred embodiment, at least part of the aqueous liquid obtained from the sulfur removing step is recycled to step a) for use as steam, for use as washing liquid and/or in the preparation of an aqueous solution of a sulfur-containing acid and/or in the preparation of an aqueous basic solution. Hence, conveniently the aqueous liquid obtained form the sulfur removing step may conveniently be recycled to for example step i), step ii) and/or step iii) as mentioned herein before. Such recycle may result in an improved water footprint of the whole of the process.

The gaseous product obtained from the process of anaerobic digestion preferably comprises methane, carbon dioxide and hydrogen sulfide. The mixture may be treated in accordance with known methods to remove hydrogen sulfide from the gaseous product, yielding a biogas comprising methane and carbon dioxide, to convert hydrogen sulfide into elemental sulfur and recover elemental sulfur. Such methods are known per se, cf. for example WO 00/53290 A1 and “Test and Quality Assurance Plan; Paques THIOPAQ and Shell Paques Gas Purification Technology”, Report prepared by Greenhouse Gas Technology Center Southern Research Institute (PO Box 13825, Research Triangle Park, North Carolina 27709, USA).

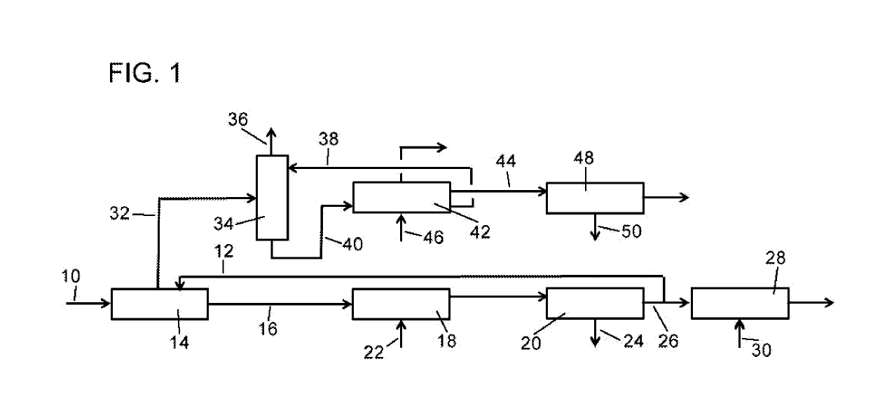

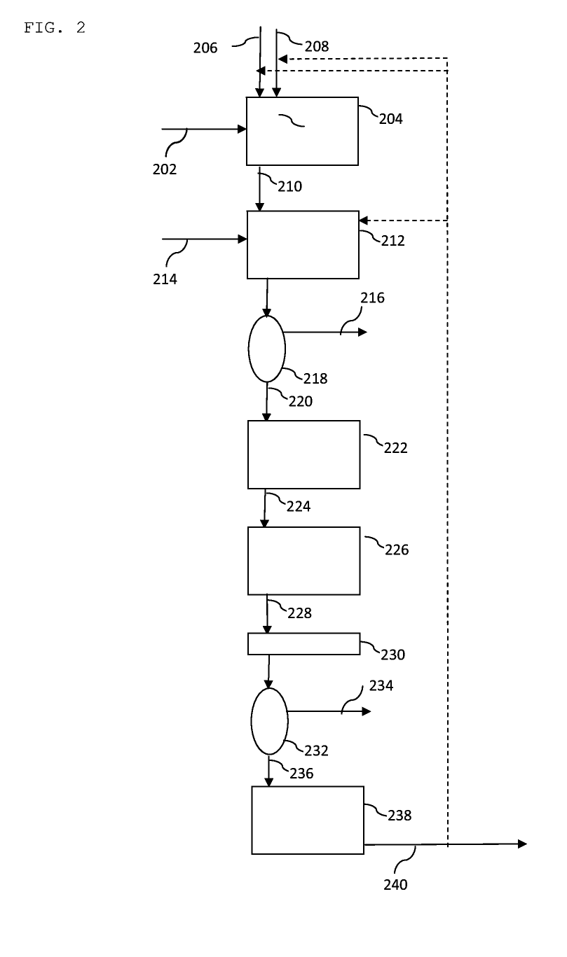

Embodiments of the present invention will now be further described with reference to FIGS. 1 and 2 and examples, all of which are intended to be illustrative and not to limit the invention.

Referring to FIG. 1, a schematic of one embodiment of a treating step according to aspects of this invention is provided. As shown, first aqueous feed (10) comprises dissolved organic materials and dissolved sulfur-containing compounds, and it is fed together with second aqueous feed (12) to reactor (14) for anaerobic digestion. The liquid product (16) obtained from reactor (14), comprising dissolved sulfur-containing compounds, is fed into a sulfur removal step comprising bioreactor (18) and separator (20). Air (22) is fed to bioreactor (18), as bioreactor (18) operates under aerobic conditions. Elemental sulfur (24) is withdrawn from separator (20). Aqueous liquid (26) obtained from the sulfur removing step may partially be employed as the second aqueous feed (12) and partially be further treated in aerobic digestion reactor (28). Air (30) is fed to aerobic digestion reactor (28). Aerobically digested aqueous liquid (54) may be obtained form aerobic digestion reactor (28). The gaseous product (32) obtained from reactor (14) may be treated in separator (34) to remove hydrogen sulfide, yielding a biogas (36). Hydrogen sulfide is removed in separator (34) by scrubbing with caustic soda (38). Sulfide rich extract (40) is treated in aerobic treater (42) to form product (44) comprising elemental sulfur. Air (46) is fed to aerobic treater (42). Product (44) is separated in separator (48). Elemental sulfur (50) is withdrawn from separator (48).

Referring to FIG. 2, there is provided a schematic of one embodiment of the conversion step according to aspects of the invention. As shown, lignocellulosic biomass (202) comprises wheat straw, and it is pretreated in pretreatment unit (204) with steam (206) and an aqueous solution of sulfuric acid (208) to produce pretreated biomass mixture (210). Pretreated biomass mixture (210) is forwarded to flasher (212), where an aqueous flash waste stream (214) is flashed off. The remaining pretreated biomass mixture (216) is washed in washing unit (217) with a stream of washing water (218), generating an aqueous wash waste stream (219) and a washed pretreated biomass mixture (220). The washed pretreated biomass mixture (220) is hydrolyzed via enzymatic hydrolysis in hydrolysis unit (222) to prepare a hydrolysis product (224). The hydrolysis product (224) is forwarded to a bioreactor (226) where it is fermented with the help of one or more microorganisms to produce a fermentation mixture containing ethanol (228). The fermentation mixture containing ethanol (228) is filtered via filter (230) and forwarded to a flasher (232), where a stream containing ethanol (234) is flashed off and an aqueous fermentation waste stream (236) is obtained. The aqueous flash waste stream (214), the aqueous wash waste stream (219) and the aqueous fermentation waste stream (236) are combined—using the aqueous flash waste stream (214) as part of a second aqueous feed and combining the aqueous wash waste stream (219) and the aqueous fermentation waste stream (236) for use as a first aqueous feed—and forwarded as a mixture to treatment unit (238). In one embodiment, the layout of treatment unit (238) is as illustrated in FIG. 1. From separator (20) of FIG. 1, an aqueous liquid (26) may be obtained and from aerobic digestion reactor (28) in FIG. 1 an aerobically digested aqueous liquid (54) may be obtained. In one embodiment, in the process of FIG. 2, part of the aqueous liquid (noted as (26) and/or (54) in FIG. 1) is obtained from treatment unit (238). This part is noted in FIG. 2 as stream (240). This aqueous liquid (240) can subsequently be at least partly recycled to pretreatment unit (204) and/or washing unit (217) for use in the preparation of steam, in the preparation of an aqueous solution of sulfuric acid and/or as washing water. These recycle streams are indicated with a dashed line in FIG. 2.

### Example 1

### According to Aspects of the Invention

A first feed is obtained as the distillation bottom product in a process in which lignocellulosic biomass is converted into ethanol and ethanol is recovered by distillation from an aqueous fermentation mixture. The first feed has been filtered to remove any solid particles and comprises glucans/glycose in a quantity of 10 g C₆H₁₂O₆/kg, xylans/xylose in a quantity of 4.7 g C₅H₁₀O₅/kg, furfural in a quantity of 1.5 g C₅H₄O₂/kg, a COD in a quantity of 20 g oxygen/kg, sulfur in a quantity of 1.63 g/kg, ammonia in a quantity of 0.5 g/kg, potassium in a quantity of 4.4 g/kg, and sodium in a quantity of 0.2 g/kg. In a continuous process, the first feed is combined with a second aqueous feed in a weight ratio of 4 kg of the second aqueous feed per kg of the first feed and the combination is fed for anaerobic digestion to an upflow anaerobic sludge blanket (UASB) reactor comprising mesophile anaerobic microorganisms comprising acetic acid forming bacteria, methane forming archaea and sulfate reducing bacteria, obtained from municipal sludge digestion. The temperature in the UASB reactor is maintained at 30° C., the pressure is atmospheric and the residence time is such that the COD is decreased by 85%.

The aqueous liquid withdrawn from the UASB reactor is fed into the bioreactor of an aerobic Shell-Paques process, comprising microorganisms of the genus Thiobacillus and microorganisms of the genus Halothiobacillus. Simultaneously with the aqueous liquid withdrawn from the UASB reactor a combination of nutrients, as specified hereinbefore, is fed into the bioreactor at a rate of at most 1.4 kg of the nutrient solution per kg of sulfide to be converted, calculated as the weight of elemental sulfur. The bioreactor is aerated by blowing air into the reactor. The temperature in the bioreactor is maintained at 30° C., the pressure is atmospheric and the average residence time of the aqueous phase is 5 hours. Elemental sulfur formed in the bioreactor is separated from the aqueous liquid by means of a gravity separator positioned inside the bioreactor.

A portion of the liquid product obtained from the bioreactor is used and recycled as the second aqueous feed, as described in this Example. As, upon recycle, a portion of the nutrients are recycled, the feeding of the combination of nutrients into the bioreactor is decreased accordingly,

After the continuous process has reached its stationary phase, the second aqueous feed comprises glucans/glycose in a quantity of 1.5 g C₆H₁₂O₆/kg, xylans/xylose in a quantity of 0.7 g C₅H₁₀O₅/kg, furfural in a quantity of 0.23 g C₅H₄O₂/kg, a COD in a quantity of 3 g oxygen/kg, and sulfur in a quantity of 0.082 g/kg, and in the UASB reactor the sulfur content of the aqueous liquid is 0.39 g/kg.

### Example 2

### According to Aspects of the Invention

Example 1 is repeated with the difference that the first feed is combined with the second aqueous feed in a weight ratio of 10 kg of the second aqueous feed per kg of the first feed, instead of 4 kg of the second aqueous feed per kg of the first feed.

After the continuous process has reached its stationary phase, the sulfur content of the aqueous liquid in the UASB reactor is 0.22 g/kg.

### Example 3

### According to Aspects of the Invention

Example 1 is repeated with the difference that the first feed comprises sulfur in a quantity of 0.57 g/kg, ammonia in a quantity of 0.55 g/kg, and potassium in a quantity of 0.44 g/kg, instead of sulfur in a quantity of 1.63 g/kg, ammonia in a quantity of 0.5 g/kg, and potassium in a quantity of 4.4 g/kg.

After the continuous process has reached its stationary phase, the sulfur content of the aqueous liquid in the UASB reactor is 0.14 g/kg.

### Example 4

### According to Aspects of the Invention

Example 3 is repeated with the difference that the first feed is combined with the second aqueous feed in a weight ratio of 2 kg of the second aqueous feed per kg of the first feed, instead of 4 kg of the second aqueous feed per kg of the first feed.

After the continuous process has reached its stationary phase, the sulfur content of the aqueous liquid in the UASB reactor is 0.21 g/kg.

### Example 5

### According to Aspects of the Invention

Example 3 is repeated with the difference that the first feed is combined with the second aqueous feed in a weight ratio of 0.5 kg of the second aqueous feed per kg of the first feed, instead of 4 kg of the second aqueous feed per kg of the first feed.

After the continuous process has reached its stationary phase, the sulfur content of the aqueous liquid in the UASB reactor is 0.39 g/kg.

### Example 6

### Comparative, not According to Aspects of the Invention

The first step of Example 1, that is the step of anaerobic digestion in the UASB reactor, is repeated with the difference that exclusively the first aqueous feed is fed to the UASB reactor, instead of feeding the combination of the first aqueous feed and the second aqueous feed. No substantial decrease in COD is found, and hence no residence time can be established such that the COD is decreased by 85%.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A process comprising the steps of:
a) converting a lignocellulosic biomass into a fuel and producing an aqueous waste stream comprising at least one dissolved organic material and at least one dissolved sulfur-containing compound, wherein the aqueous waste stream has a sulfur content of more than 400 parts per million by weight, relative to the weight of the aqueous waste stream; and
b) treating the aqueous waste stream, said treating comprises anaerobic digestion of a mixture comprising a first aqueous feed comprising the aqueous waste stream and a second aqueous feed, wherein the mixture has a sulfur content of at most 400 parts per million by weight, relative to the weight of the mixture.

2. The process of claim 1, wherein the second aqueous feed comprises at least one dissolved sulfur-containing compound and has a sulfur content of less than 400 parts per million by weight, relative to the weight of the second aqueous feed.

3. The process of claim 1 wherein the second aqueous feed does not comprise a dissolved sulfur-containing compound and does not have a sulfur content.

4. The process of claim 1, wherein the dissolved organic materials comprise one or more of alcohols, monosaccharides, disaccharides, oligosaccharides, polysaccharides, aldehydes, vegetable oils, and volatile vegetable acids.

5. The process of claim 1, wherein the first aqueous feed has a Chemical oxygen demand of at most 10⁵ mg oxygen per liter of the first aqueous feed.

6. The process of claim 5 wherein the Chemical oxygen demand is in the range of from 5×10³ mg oxygen per liter of the first aqueous feed to 8×10⁴ mg oxygen per liter of the first aqueous feed.

7. The process of claim 5 wherein the chemical oxygen demand is in the range of from 1×10⁴ mg oxygen per liter of the first aqueous feed to 7×10⁴ mg oxygen per liter of the first aqueous feed.

8. The process of claim 1, wherein step a) comprises breaking up of the lignocellulosic biomass using sulfuric acid and the at least one dissolved sulfur-containing compound present in the first aqueous feed comprises sulfates and/or sulfuric acid.

9. The process of claim 1, wherein the sulfur content of the first aqueous feed is in the range of from 450 parts per million by weight to 4000 parts per million by weight, relative to the weight of the first aqueous feed.

10. The process of claim 9 wherein the sulfur content of the first aqueous feed is in the range of from 500 parts per million by weight to 3000 parts per million by weight, relative to the weight of the first aqueous feed.

11. The process of claim 1, wherein the step of converting the lignocellulosic biomass into a fuel comprises:
pretreating the lignocellulosic biomass with an aqueous solution of a sulfur-containing acid and optionally with steam to produce a pretreated biomass mixture; and
flashing the pretreated biomass mixture to remove water to produce a flashed pretreated biomass mixture and an aqueous flash waste stream;
wherein the aqueous flash waste stream has a sulfur content, if any, of less than 400 parts per million by weight, relative to the weight of the aqueous flash waste feed and is used as at least part of a second aqueous feed in step b); and
wherein the aqueous wash waste stream comprises dissolved organic materials and dissolved sulfur-containing compounds, and has a sulfur content of more than 400 parts per million by weight, relative to the weight of the aqueous wash waste stream and is used as at least part of a first aqueous feed in step b).

12. The process of claim 11 further comprising the step of: washing and/or neutralizing the pretreated biomass mixture with water and/or an aqueous basic solution to produce a washed pretreated biomass mixture and an aqueous wash waste stream.

13. The process of claim 1 wherein the step of converting the lignocellulosic biomass into a fuel comprises:
pretreating the lignocellulosic biomass with an aqueous solution of a sulfur-containing acid and optionally with steam to produce a pretreated biomass mixture; and
washing and/or neutralizing the pretreated biomass mixture with water and/or an aqueous basic solution to produce a washed pretreated biomass mixture and an aqueous wash waste stream.

14. The process of claim 2, wherein the sulfur content is at most 300 parts per million by weight, relative to the weight of the second aqueous feed.

15. The process of claim 2, wherein the sulfur content is more preferably at most 200 parts per million by weight, relative to the weight of the second aqueous feed.

16. The process of claim 2, wherein the second aqueous feed comprises sulfide salts and/or hydrogen sulfide.

17. The process of claim 1, wherein the Chemical oxygen demand of the second aqueous feed is at most 1×10⁴ mg oxygen per liter of the second aqueous feed.

18. The process of claim 17, wherein the Chemical oxygen demand is at most 8×10³ mg oxygen per liter of the second aqueous feed.

19. The process claim 1 further comprising feeding the first aqueous feed and the second aqueous feed in a relative proportion to provide a sulfur content of the resultant mixture of at most 390 parts per million by weight.

20. The process of claim 19 wherein the sulfur content is at most 380 parts per million by weight.

21. The process of claim 1 further comprising feeding the first aqueous feed and the second aqueous feed in a relative proportion to provide a weight of the second aqueous feed relative to a weight of the first aqueous feed in a range of from 20 to 0.1.

22. The process of claim 21 wherein the range is from 15 to 0.25.

23. The process of claim 1, wherein the mixture comprises one or more salts of sodium, potassium, magnesium, ammonium, and any combination thereof.

24. The process of claim 23, wherein the one or more salts has a sodium content in a range of from 50 parts per million by weight to 8000 parts per million by weight, relative to the weight of the mixture.

25. The process of claim 24 wherein the range is from 100 parts per million by weight to 5500 parts per million by weight, relative to the weight of the mixture.

26. The process of claim 23, wherein the one or more salts has a potassium content a range of from 100 parts per million by weight to 12000 parts per million by weight, relative to the weight of the mixture.

27. The process of claim 26, wherein the range is from 200 parts per million by weight to 5000 parts per million by weight, relative to the weight of the mixture.

28. The process of claim 23, wherein the one or more salts has a magnesium content in the range of from 40 parts per million by weight to 3000 parts per million by weight, relative to the weight of the mixture.

29. The process of claim 28, wherein the range is from 75 parts per million by weight to 1500 parts per million by weight, relative to the weight of the mixture.

30. The process of claim 23, wherein the one or more salts has a content of ammonium salts in a range of from 25 parts per million by weight to 4000 parts per million by weight, relative to the weight of the mixture, wherein the content of ammonium salts relates to the quantity of ammonium salts calculated as the weight of the NH₄ moiety.

31. The process of claim 30, wherein the range is from 50 parts per million by weight to 3000 parts per million by weight, relative to the weight of the mixture, wherein the content of ammonium salts relates to the quantity of ammonium salts calculated as the weight of the NH₄ moiety.

32. The process of claim 1, wherein the anaerobic digestion yields an aqueous liquid product and wherein the process further comprises a step of removing at least a portion of the at least one dissolved sulfur-containing compound from the aqueous liquid product.

33. The process of claim 32, wherein the removing step comprises treating the aqueous liquid product in an aerobic process.

34. The process of claim 32, wherein the removing step yields an aqueous liquid and wherein the process further comprises recycling at least a portion of the aqueous liquid as the second aqueous feed, or as a portion of the second aqueous feed.
